(19) United States
(12) Patent Application Publication
(10) Pub. No.: US 2010/0144006 A1
(43) Pub. Date: Jun. 10, 2010

(54) Sample Concentration Method and Apparatus

(76) Inventor: Stanley E. Charm, Boston, MA (US)

Correspondence Address: CHARM SCIENCES, INC., 659 ANDOVER STREET, LAWRENCE, MA, 01843, US

(21) Appl. No.: 12/703,953

(22) Filed: Feb. 11, 2010

Related U.S. Application Data
(63) Continuation-in-part of application No. PCT/US2008/009706, filed on Aug. 14, 2008.
(60) Provisional application No. 60/955,761, filed on Aug. 14, 2007.

(51) Int. Cl. C12N 13/00; C12M 1/00

(52) U.S. Cl. 435/173.9; 435/283.1

(57) ABSTRACT

A method and apparatus for controlled concentration of an analyte containing liquid sample. Embodiments include using microwave energy to heat a sample while reducing pressure in a chamber. The combination of reduced pressure and microwave energy can provide sufficient heat to vaporize a portion of the liquid while maintaining analyte integrity. The method and apparatus can increase speed and sensitivity of analyte detection.

### REFERENCE TO PRIOR APPLICATION

This application is based on and claims priority to and is a continuation-in-part of PCT/US2008/009706, filed Aug. 14, 2008, which further claims priority to U.S. Provisional Patent Application No. 60/955,761, filed on Aug. 14, 2007, the teachings of both of which are incorporated herein by this reference.

### FIELD

The invention relates generally to an apparatus and method for sample concentration, and more particularly, to a concentration system that utilizes both heat, for example heat generated using microwave energy, and a vacuum.

### BACKGROUND

When detecting microorganisms or other biological material, it is often helpful to reduce the volume of the solvent. By reducing the volume of the solvent without a corresponding reduction in the amount of the material to be detected, detection sensitivity and detection speed can both be enhanced. For example, when detecting bacteria or bacteriophage (phage) in a solvent such as water, concentrating the sample while maintaining the number and viability of the bacteria or phage, can increase the sensitivity of the detection mechanism. Similarly, in polymerase chain reaction technology, reducing sample volume without reducing the amount of DNA can increase the sensitivity of detection.

The microwave is now a common commercially-available apparatus and microwave heating of various materials to dry, evaporate, effect chemical reactions, and application in other various laboratory purposes, is well known. The microwave apparatus offers rapid results and, therefore, its use is carried out routinely in a variety of manufacturing processes. The conventional procedure of using microwave energy for elevating the temperature of a sample is, however, not ideal for the controlled concentration of a heat sensitive sample.

### SUMMARY

Aspects include a method and apparatus for concentrating an analyte containing sample without degrading the analyte. The methods and apparatuses described herein can be useful with a variety of analytes that may be present within a solvent, such as water, including phage, microbes, proteins and nucleic acids. Aspects include the steps of: placing a container, within which is a sample, into a chamber, such as a gas impermeable chamber that can act as a vaporization/concentration chamber; applying heat, for example through application of controlled microwave energy, to the sample to vaporize a solvent from the sample; subjecting the sample to a controlled pressure, through a gas/vapor exhaust system by removing air from an outlet, to speed concentration and reduce the temperature at which vaporization can occur; vibrating the sample; and terminating the concentration process when the measured concentration reaches a predetermined target value. When microwaves are used as the heat source, the chamber can be made of microwave permeable material. Sample can be vibrated, for example by agitating the sample using a rotating turntable support assembly that is controlled by a motor. The motor can be capable of rotating the turntable both clockwise and counterclockwise. Advantages include the ability to control concentration of a sample, for example by reducing the microwave energy as the sample is concentrated. Such controlled concentration can both prevent the sample from freezing and overheating. Pressure can be controlled to provide an environment in which solvent is evaporated at a reduced temperature and complete vaporization of the liquid is prevented. To control the air pressure a vacuum pump can be used. A condenser can be situated between the microwave source and the vacuum pump to prevent some or all the vapor from traveling to the pump. Excess vapor can be captured by a trap. Non-condensable vapor can be captured by a filter, such as a filter that is both a HEPA filter and a coalescing filter. All or part of the controls can be electronic.

Additional aspects include providing a concentrating apparatus that is compact in size for use in a laboratory setting, with limited space. Yet another aspect is to provide an apparatus configured to reduce splattering of the sample during the concentration process. Another aspect includes optimizing the configuration and/or composition of the chamber, for example by adding material to the chamber side-wall or changing the configuration of a portion of the chamber side-wall to optimize the usage of the available microwave energy. For example, certain material when added to the chamber side-wall may focus and/or capture the microwave radiation so that it is available in the desired areas to enhance the concentration efficiency.

These and other aspects of the present invention will become apparent to those skilled in the art after seeing the following drawings and written description.

### BRIEF DESCRIPTION OF THE DRAWINGS

FIG. 1 is a front-perspective view of an embodiment of the disclosure.

FIG. 2 is a front-perspective view of an embodiment of the disclosure with microwave door open and chamber 4 being exposed.

FIG. 3 is a partial front-perspective view of an embodiment of chamber 4.

FIG. 4 is a top-view of the interior of chamber 4, which comprises a plurality of containers 12.

FIG. 4A is a top vertical sectional view of an embodiment of container 12, which comprises a sample solution 200;

FIG. 5 is a top-view of the interior of the chamber, which comprises a plurality of containers 12; and

FIG. 5A is a top vertical sectional view of an embodiment of container 12, which comprises a concentrated solution 202.

FIG. 6 is a front-perspective view of an embodiment of the disclosure with microwave door open and chamber 4 being exposed showing changes in the chamber side-wall 300 configuration by the additional material 110. Also shown is supporting structure 100 with holes 101 to enhance the gas removal capability.

FIG. 7 is a top vertical sectional view of an embodiment of chamber 4 with addition of microwave manipulating material 110 to side-wall 300 of chamber.

### DETAILED DESCRIPTION

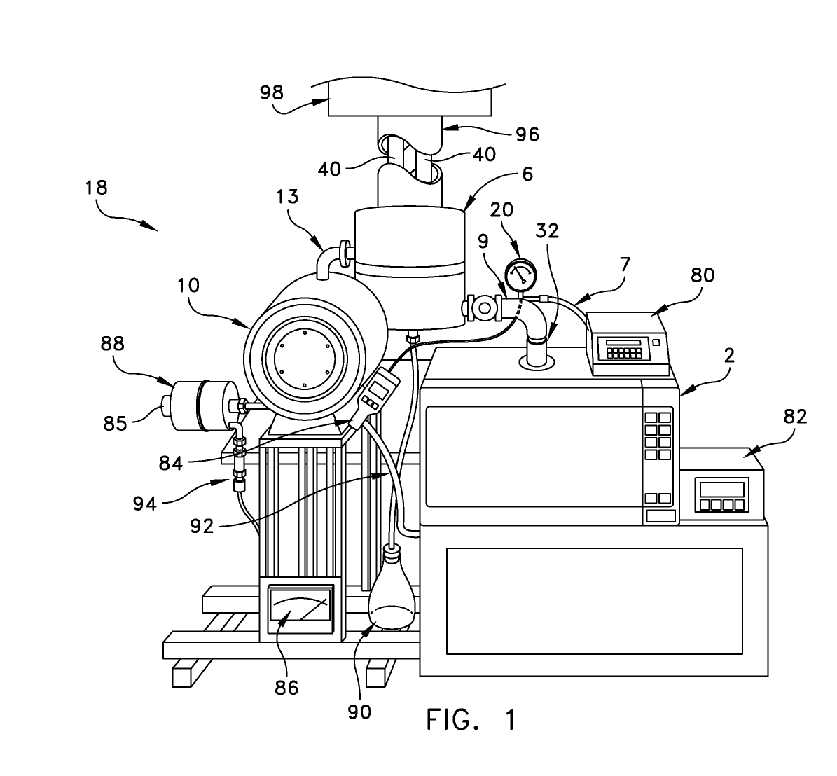

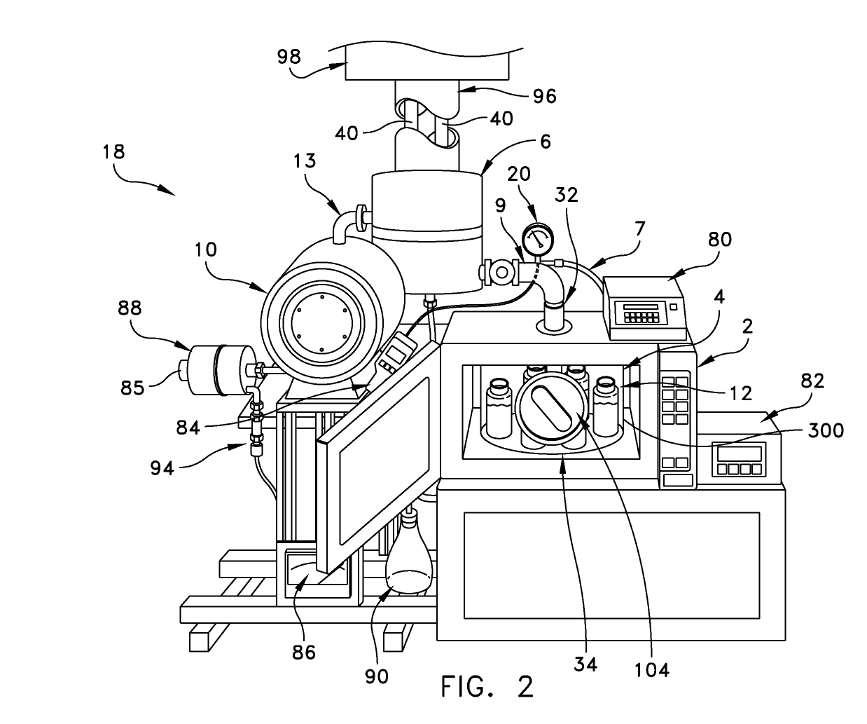

The following description may include like reference characters that correspond to like elements throughout the several figures. The terms ‘left,’ ‘right,’ ‘forward,’ ‘rearward,’ and the like are words of convenience to describe various embodiments and should not be construed as limitations to the scope of the invention. Referring now to the drawings in general and FIGS. 1 and 2 in particular, the views are for the benefit of describing an embodiment of the invention and are not intended to limit the scope of the invention.

Embodiments include a method and apparatus for controlled concentration of a liquid sample thought to contain an analyte. In an example, microwave energy is applied to a microwave permeable, gas impermeable chamber—a concentration chamber—within which a sample has been placed. In cooperation with the application of microwave energy, a vacuum pump reduces pressure in the concentration chamber. The combination of reduced pressure and microwave energy can provide enough heat to vaporize a portion of the liquid while not providing so much heat that the analyte is destroyed or denatured. The method and apparatus can increase speed and sensitivity of analyte detection.

As shown in the figures the concentration process includes placing the sample within concentration chamber 4, reducing pressure in chamber 4 with a vacuum pump 10, and applying microwaves from microwave 2 to contents of chamber 4 to concentrate liquid solution 200 from sample 14. The combination of heat and reduced pressure cause vaporization of liquid solution 200 at non-destructive temperatures for a heat-sensitive analyte. By non-destructive temperatures we mean a temperature which if applied to an analyte for a particular predetermined period of time will not destroy or denature the analyte.

In an embodiment, sample container 12 is movably repositioned by a turntable assembly 34 that is alternatively moved clockwise and counterclockwise by a motor attached to turntable assembly 34. The clockwise and counterclockwise movement vibrates the sample sufficiently to limit or eliminate splattering when the sample is heated. As described herein, vibrating the sample includes any manner of oscillating, shaking, quivering or otherwise moving the sample. As described herein, splattering of the sample and/or sample solvent includes any boiling, bubbling or explosive effect that might cause liquid from the sample container to leave the container.

Sample 14 is placed within at least one of a plurality of sample containers 12, which is then placed into chamber 4 within microwave heater 2. Non-sample solution can be provided, for example in a volume larger than the sample volume, to prevent arcing within the microwave when the sample volumes become so low as to create an environment in which arcing may otherwise occur.

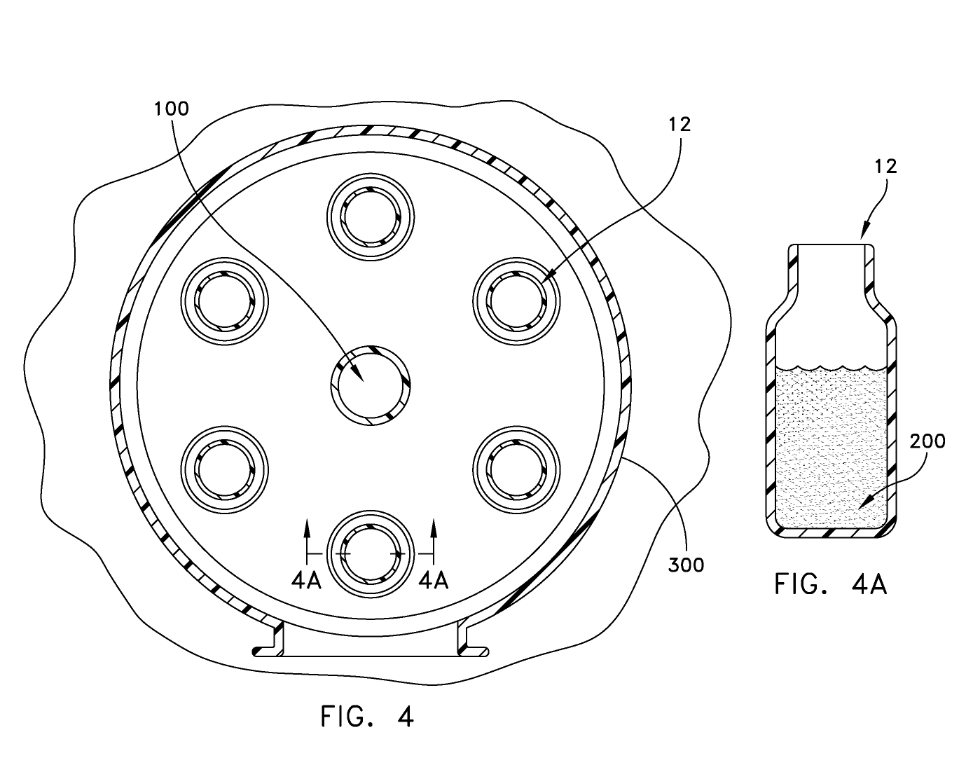

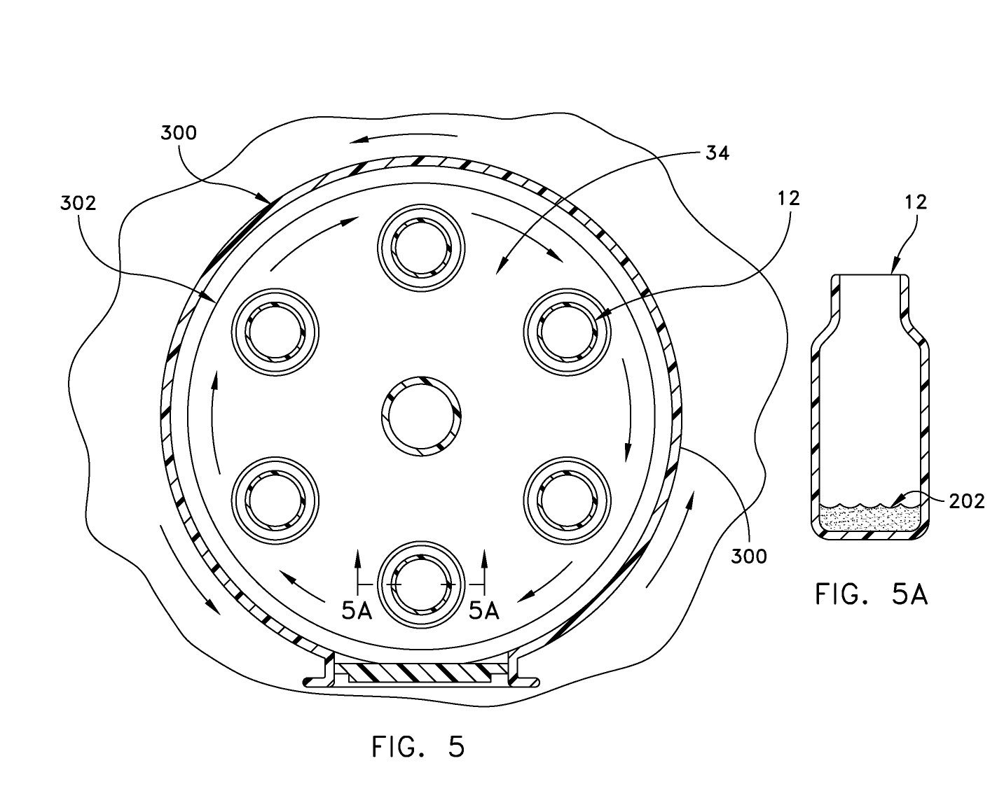

A plurality of containers 12 can be configured in a variety of shapes and sizes, including incorporating a fully-exposed open top, a partially-exposed top, or a closed top. When samples are thought to contain pathogens, at least a partial enclosure of the containers may be desirable, for example utilizing a gas permeable plug such as a foam plug or a cotton plug. As seen in FIG. 4A, sample 14 may comprise a liquid solution 200 in container 12 prior to, or during, the initial stages of concentration. FIG. 5A, illustrates container 12 after operation of sample concentrator 18, where sample 14 has been concentrated, where the quantity of liquid solution 200 is reduced as illustrated by solution 202.

Container 12 used to retain sample 14 can be designed to prevent an analyte, such as a bacteria or phage, from adhering on the wall of the sample container during and after the liquid is vaporized. For example, silanization can be used to coat the wall of the sample container to prevent adhesion of bacteria to the inner-wall surface of the container. The shape of the container used can similarly be optimized to prevent analyte concentration on the walls and/or for convenient sample pooling after concentration. For example, a cone shaped container can be used so that as liquid or similar solvent is removed from the sample, and the sample is concentrated, the sample will be at the bottom of the cone where the surface area is smaller. Finally, for increased productivity in a laboratory setting, the container 12 can be autoclavable. It has been found that glass containers, such as PYREX (PYREX is a registered trademark of Corning Glass Works Corporation, New York) containers are particularly useful as compared to, for example plastic containers, to prevent splattering. Although not wishing to be constrained by theory, it may be that, as compared to plastic or other materials, glass limits nucleation and the resultant boiling and splattering.

As seen in FIG. 1, sample container 12 is positioned inside of chamber 4, which is positioned in the interior of microwave 2. The assemblage of chamber 4 to microwave 2 can be a fixed connection, a freely removable connection, or a combination thereof. Chamber 4 can be removably connected to the base of microwave 2 to allow removal for cleaning and maintenance. In a further embodiment, chamber 4 is affixed to the floor or base of turntable assembly 34. Chamber 4 can be made from a microwave permeable and gas impermeable material, including: plastic, quartz, ceramics, or a like material. This is particular useful when using microwaves as a source of heat. Chamber 4 can have an inlet to allow suction generated from vacuum pump 10 to reduce pressure within chamber 4 and allow rapid concentration of sample 14.

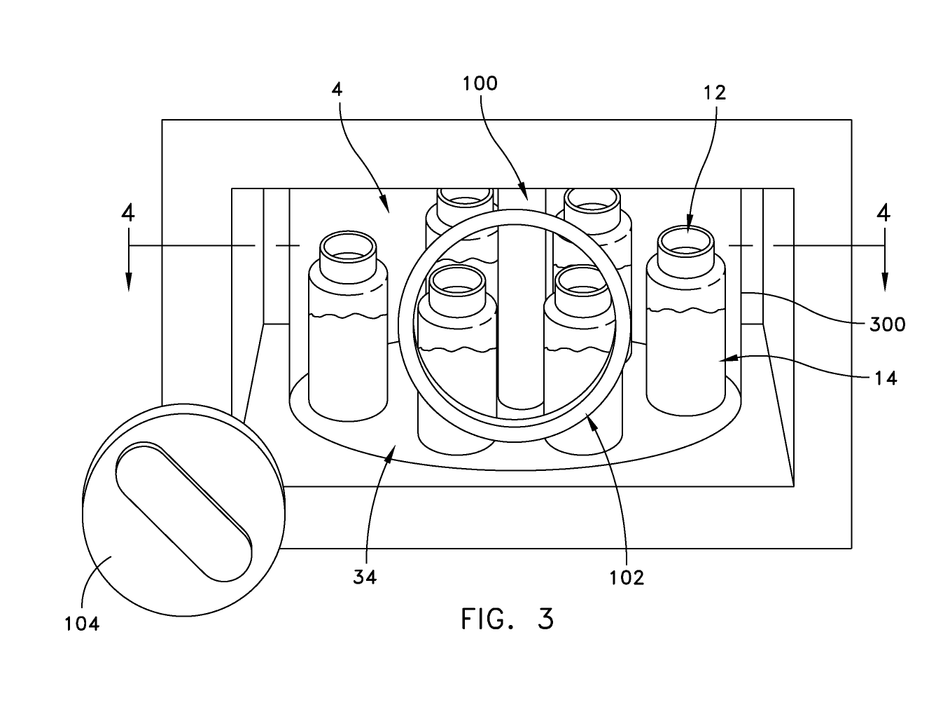

As seen in FIGS. 2 and 3, chamber 4 may further comprise chamber closure 104. In such an embodiment, chamber closure 104 exposes containers 12 for maintenance and handling. Chamber closure 104 may be configured to seal chamber opening 102. Chamber closure 104 can be made of a microwave permeable, gas impermeable material, for example acylic, polycarbonate or high density polyethylene, to provide a chamber that allows rapid concentration of sample 14 at predetermined rate. Chamber opening 102 is sized according to the particular dimensions of chamber closure 104 and containers 12 being serviced.

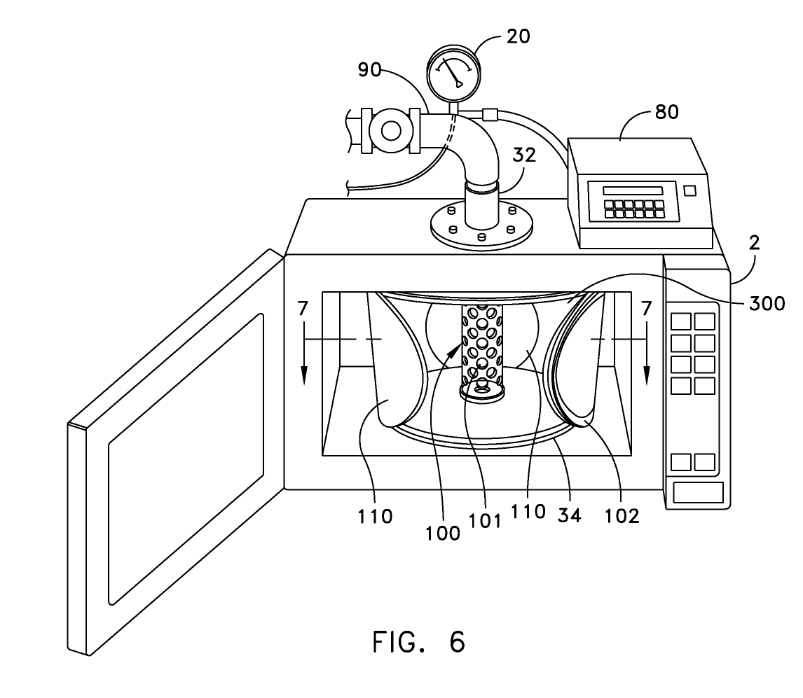

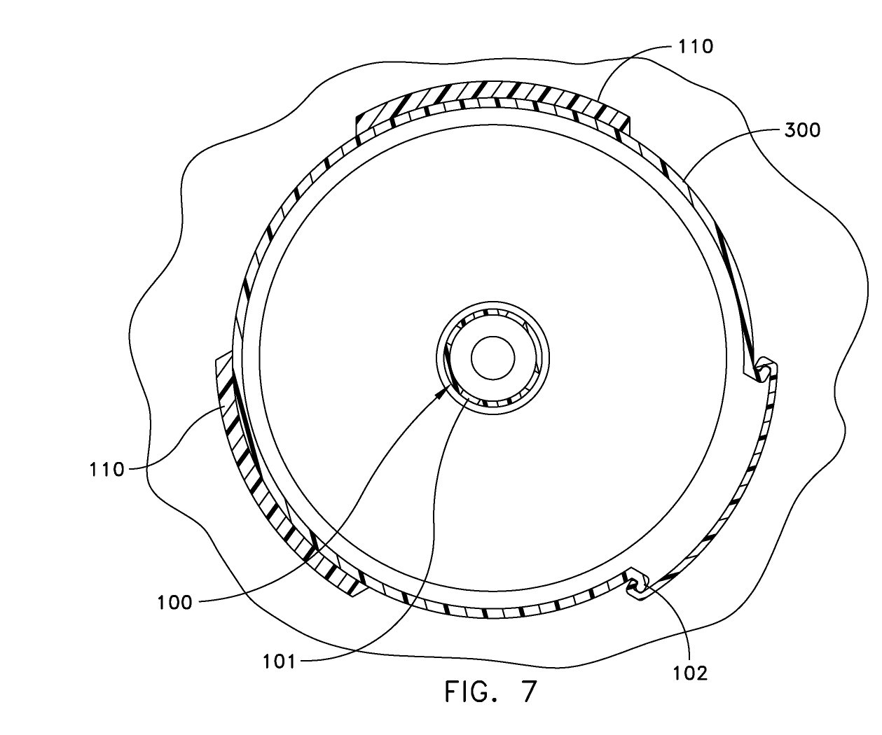

Chamber 4 has top, bottom and side-wall 300 and an inlet through which air and vapor can be removed. Perimeter of chamber 4 can be adapted with a sealing gasket to maintain a vacuum. When pressure is reduced within chamber 4 it may be necessary to provide supporting structure 100 within chamber 4 to prevent collapse. Supporting structure 100 can comprise a microwave permeable material and can be positioned in a variety of locations within the chamber. For example, supporting structure 100 can be positioned below the vacuum inlet, in which case it can have holes to allow gas permeability. It can also be positioned off of the center of the turntable and/or chamber. Supporting structure 100 can also include holes (as shown in FIGS. 6 and 7) to enhance gas removal.

As seen in FIGS. 1 and 2, vacuum pump 10 is activated to reduce the pressure within chamber 4 to speed vaporization. Condenser 6 includes tubing in communication with vacuum pump 10. In some embodiments, refrigerant tubing 40 is in communication with condenser 6 and a refrigeration unit (not shown) and directs flow of coolant into said condenser 6 to maintain condenser temperature, for example in the range of about minus 135° F. to about minus 142° F. Similarly, condenser 6 is positioned between vacuum 10 and microwave 2 to condense sample vapor leaving the sample containers 12. By condensing sample vapor before it enters vacuum pump 10, efficiency of vacuum pump 10 is maintained. Vapor may nevertheless enter the vacuum pump 10. Filter 88 can include a vapor/water separator so that water, or other liquid, is removed through line 94 to a trap. Vapor can be filtered before it is removed from the system through exhaust 85. Filter 88 can be, for example, a filter with combined capability to act as a HEPA filter and a coalescing filter.

Condenser 6 can be designed for maximum surface area to enhance heat transfer and, therefore, the vaporization efficiency. Rapid thawing of condenser 6 may be important so that the system can be rapidly restored and prepared for multiple sample concentration procedures. To effectuate such rapid thawing, condenser 6 can include heating coils. Such heating cools can be adapted for controlled heating so as not to damage coolant. If rapid thawing is not required, for example if reconditioning of coolant is sufficient, heating coils might not be required.

Electronic controls provide sufficient control over heat, rotation, and vacuum in chamber 4 to concentrate sample 14 to a desired end-point. In some embodiments, electronic controls may be regulated by a microprocessor digital computer or a programmable analyzer. Such configuration allows concentration of sample 14 to occur at predetermined stages including a predetermined initial concentration stage, followed by successive reduced concentration. The electronic controls can include turntable control 82, vacuum actuator control 84, and microwave controls which alone or in combination, maintain a desired environment to prevent destruction, including freezing, overheating and drying. For example, when detecting a bacteria or phage, it is important for the temperature to be optimized to maintain viability. Useful operating temperatures can be in the range of about 5° C. to about 15° C. Similarly, temperature and/or pressure controls are required to prevent splattering of sample out of container 12. Turntable control 82 can be used to control the speed and period of rotation, both clockwise and counterclockwise, to vibrate the sample to prevent splattering. In an example, turntable 34 is rotated by a motor that can rotate turntable 34 both clockwise and counterclockwise. The motor can be connected directly or indirectly to turntable 34 or can be positioned in another position, so long as it can function to rotate turntable 34 clockwise and counterclockwise. Such turntable motor can be located in a variety of positions relative to chamber 4.

Embodiments herein describe vibrating the sample through use of the rotatable turntable 34 upon which sits chamber 4. Other methods can also be used. For example, the chamber can sit within the microwave on a pivot controlled by a motor. A pivot can be attached to the chamber or to a platform upon which the chamber sits. In those embodiments a sample can be vibrated in not only a turning motion but also a rocking motion. Still another embodiment includes applying ultrasonic waves to the sample to vibrate the sample.

In an example, vacuum actuator controller 84 was a J-KEM Scientific Infinity Controller (J-KEM is a registered trademark of J-KEM ELECTRONICS, INC. St. Louis Mo.). The vacuum actuator controller 84 can be preset for automated ramp-to-setpoint control or be set manually. Air pressure can be monitored at various locations, for example with pressure gauges 20, 84 and 86.

In an example, the turntable was connected to a brushless servo motor connected to a servo drive. The unit operated in pulse follower mode. Pulses that determined the direction and speed of the turntable were generated by a micro Programmable Logic Controller (PLC.). The pulse settings were fed to the PLC from an Operator Interface Terminal (OIT) that allowed the operator to input speed, time and direction of motion. The turntable could also be operated manually through the OIT. The turntable was designed to alternate between the programmed forward and reverse movements. The back and forth movement vibrates the sample.

A range of turntable control settings can be usefully employed. For example, the control can be set to move the turntable 25 revolutions per minute (RPM) in one direction and then 20 RPM in the opposite direction so that net rotation of the turntable was 5 RPM. Similarly, the controls can be set to move the turntable 25 RPM in one direction for a period of time and in counterclockwise direction, at the same RPM, for a shorter period of time. In either example, a net forward (clockwise) rotation is obtained. The net forward rotation can be useful for consistent heating of the sample within the microwave field but is not required.

An inlet into the chamber can be used as an inlet for the vacuum pump tubing 32. That inlet can also serve as the outlet for vapor from the chamber. When vacuum pump 10 pulls air from chamber 4, water or solvent molecules from chamber 4 can be swept out of chamber 4 before condensing. Between vacuum pump 10 and chamber 4 can be condenser 6 where the vapor can condense and flow into container 90. Container 90 can also be used to capture liquid removed from condenser 10, such as after defrosting of condenser 10. Condenser 6 can be cooled such as, for example, with liquid nitrogen or cooling fluid from a source such as a refrigeration unit. In some embodiments cooling temperatures can be controlled electronically with a microprocessor digital computer.

In a laboratory setting, with limited space, the compactness of the system is particularly important. A variety of microwave sources can be used including those of the dimensions of a standard home kitchen microwave.

In some embodiments, a rotating, shaking turntable can be mounted on the bottom of the microwave and hold the concentration chamber. Turntable 34 can be made of a material to allow dissipation of heat to prevent the overheating of the bottom of chamber 4. The thickness of turntable 34 and other features can be varied to influence the impact of the microwaves in chamber 4.

To concentrate the sample, the liquid sample is placed within at least one of the sample containers and then into chamber 4 within a microwave heater 2. Non-sample solution can be present to help prevent microwave arcing. A vacuum pump 10 is activated to reduce the pressure within chamber 4 and to speed and sensitize vaporization. Vacuum pump 10, via tubing 8 into chamber 4 also helps pull the vapor into condenser 6.

As seen in FIG. 5, turntable 34 can rotate both clockwise and counterclockwise to allow the sample to have a rocking and shaking movement and, thereby, to suppress splattering of the sample.

As seen in FIGS. 6 and 7, chamber 4 can be designed to manipulate the microwave to affect what occurs within chamber 4. For example, additional material 110 can be added to change the chamber side wall 300 configuration. Such changes in side wall 300 configuration can be useful to manipulate the microwaves, such as, for example, by focusing, capturing or otherwise manipulating the microwaves, for example in a lens-like manner, to increase the relative speed of sample concentration or otherwise enhance concentration efficiency and microwave power usage efficiency. Additional material 110 can be composed of a plastic material including thermoplastics. Examples of useful plastics include acrylic, polycarbonate and high density polyethylene. Alternatively, rather than adding material to the outside of the chamber, areas of the chamber can be removed and replaced by a window composed of microwave manipulating materials. Although not wanting to be constrained by theory, the concentration efficiency enhancement may occur due to the focusing and/or capturing of microwaves by additional material 110. Similarly, the thickness of the additional material 110, the side-wall 300 and/or the additional material 100 may affect the microwave manipulation. For example, in embodiments, the chamber closure 104 is thicker than the additional material 110. Concentration efficiency differences can be observed in samples situated adjacent the chamber closure 104 as compared to samples situated adjacent the additional material and/or the chamber wall.

Additional aspects as shown in FIG. 6 include providing holes 101 in support 100 to allow the vacuum to exhaust through support 100.

In a specific example in the chamber configuration of FIGS. 1-5, two baffled, glass flasks containing 100 milliters (mL) of water sample were concentrated. In addition to the two baffled flasks a beaker containing 200 mL water (a blank) was included within the microwave to prevent arcing. A 1300 watt microwave was used and set to 40% power for the first 10 minutes of concentrating and 50% power to the remaining 21.5 minutes. Condenser temperature was maintained at approximately minus 135° F. to about minus 142° F. Final volumes were 7.1 mL, 6.6 mL and 55 mL. The vacuum controller initially (within the first 30 seconds) reduced the pressure within the sample chamber from 760 millitorr to 50 millitorr. Pressure was reduced to a final pressure of 5 millitorr in the following sequence: Time in seconds (T) 0/Pressure in millitorres (P) 760; T 30/P50; T 60/P25; T 90/P20; T120/P20; T155/P18; T180/P16; T205/P16; T230/P14; T255/P12; T280/P10; T305/P10; T350/P8; T395/P6; T440/P5; T500/P5. Controls were set to move 23 RPM forward (clockwise) for 1.0 second and 23 RPM in reverse (counterclockwise) for 0.8 seconds. After the above sequence, pressure was maintained at P5 for the remainder of the concentration run. Final volumes of 7.1 mL, 6.6 mL and 55 mL were reached at approximately T1860.

In another example 8 baffled, glass flasks containing 100 mL of deionized water sample were concentrated. A 1200 watt microwave was used and set to 60% power for the first 43 minutes of concentrating 50% power for the next 5 minutes and 4% power for the remaining 5 minutes. Other control parameters were as in the previous example described in paragraph [0040]. Final volumes ranged from 1.5 mL to 5.0 mL. The 1.5 mL volume was adjacent the chamber closure 104.

Several embodiments and advantages of the concentrator apparatus and method have been set forth in the foregoing description and many of the novel features are captured in the following claims. The disclosure, however, is illustrative only, and modifications by one of skill in the art may be made with the present specification and drawings without departing from the invention.

1. An apparatus for concentrating a sample comprising:
a) a microwave source, said microwave source having a base and a perimeter to maintain a microwave, the perimeter comprising a side-wall;
b) a microwave permeable chamber, said chamber having a port and a perimeter configured to maintain a vacuum flow and support a plurality of sample containers at least one of which includes a sample;
c) a motor assembly for moving vibrating the sample;
d) a vacuum pump in communication with the chamber port; and
e) a condenser positioned to condense a vapor that exits the chamber through the chamber port.

2. The apparatus of claim 1 further comprising a rotatable turntable attached to the microwave base and supporting the chamber.

3. The apparatus of claim 2 wherein said motor assembly is capable of rotating the turntable both clockwise and counterclockwise in order to vibrate the sample.

4. The apparatus of claim 1, wherein said condenser comprises a front port and a rear port, said front port being in communication with the chamber and said rear port being in communication with a refrigeration unit.

5. The apparatus in claim 1, further comprising a plurality of tubing interconnecting the condenser to the refrigeration unit and supporting the flow of coolant liquid from the refrigeration unit to the condenser.

6. The apparatus in claim 1, further comprising a chamber opening located with the perimeter side-wall, providing access to the containers and a chamber valve closure, said chamber valve closure removably affixed to the chamber valve opening.

7. The apparatus in claim 1 further comprising a trap between the vacuum pump and the port, the trap configured to condense vapor exiting the chamber through the port.

8. The apparatus in claim 1 further comprising electronic controls configured to prevent complete vaporization of the liquid.

9. The apparatus of claim 1 wherein the motor assembly is connected to the chamber.

10. The apparatus of claim 1 wherein the analyte sample is a bacteriophage.

11. The apparatus of claim 1 wherein the analyte sample is a bacteria.

12. The apparatus of claim 1 wherein the side-wall further comprises additional areas of material configured to manipulate the microwaves in order to optimize sample concentration.

13. The apparatus of claim 12 wherein the material within the additional areas of material comprises a plastic.

14. An apparatus for concentrating a sample comprising:
a) a microwave source, said microwave having a base and a perimeter to maintain a microwave;
b) a microwave permeable, gas impermeable chamber, said chamber having a port and a perimeter configured to maintain a vacuum flow and support a plurality of sample containers at least one of which includes a sample;
c) a motor assembly, the motor assembly configured to vibrate the sample by alternately rotating a turntable in a clockwise and counterclockwise direction, the turntable attached to the microwave base and supporting the chamber;
d) a vacuum pump in communication with the chamber port; and
e) a condenser positioned to condense a vapor that exits the chamber through the chamber port.

15. The apparatus of claim 14 further comprising electronic controls, the electronic controls configured to reduce the microwave energy as the sample is concentrated.

16. The apparatus of claim 14 further comprising electronic controls, the electronic controls configured to subject the sample to a controlled pressure by varying the pressure as the sample is concentrated to prevent both destruction of the analyte and complete vaporization of the liquid.

17. The apparatus of claim 14 wherein the chamber further comprises areas of microwave focusing material, said material comprising a plastic.

18. A method of concentrating an analyte containing sample without degrading the analyte comprising the steps of:
a) placing a container, within which is a sample, in a chamber comprising a side-wall, the chamber characterized by its microwave permeability and gas impermeability;
b) applying a controlled microwave energy to the sample to vaporize a solvent from the sample;
c) subjecting the sample to a controlled pressure through a gas exhaust system by removing air from an outlet to sensitize and speed concentration;
d) vibrating the sample; and
e) terminating the concentration process when the measured concentration reaches a predetermined target value.

19. The method of claim 18 wherein vibrating the sample comprises rotating a turntable support assembly in an alternating clockwise and counterclockwise motion.

20. The method of claim 18 wherein vibrating the sample comprises shaking the sample.

21. The method of claim 18 wherein vibrating the sample comprises rotating the chamber.

22. The method of claim 18 wherein the analyte comprises a bacteriophage.

23. The method of claim 18 wherein the analyte comprises bacteria.

24. The method of claim 18 wherein the step of applying the microwave energy comprises reducing the microwave energy as the sample is concentrated.

25. The method of claim 18 wherein the step of applying the microwave energy comprises varying the microwave energy to prevent the sample from freezing, while limiting a temperature increase to prevent overheating the sample.

26. The method of claim 18 wherein the step of subjecting the sample to a controlled pressure comprises varying the pressure as the sample is concentrated to prevent a destruction of the analyte.

27. The method of claim 18 wherein the step of subjecting the sample container to a controlled pressure includes using a vacuum pump to remove a vaporizable liquid from the sample.

28. The method of claim 18 wherein the chamber side-wall comprises areas of material configured to manipulate microwaves in order to speed sample concentration.

29. The method of claim 18 wherein the chamber side-wall further comprises areas of material configured to manipulate microwave in order to speed sample concentration, said material comprising a thermoplastic.
